(19) United States
(12) Patent Application Publication
(10) Pub. No.: US 2022/0203085 A1
(43) Pub. Date: Jun. 30, 2022

(54) INTRACORPOREAL GUIDE COMPONENT

(71) Applicant: CORLIFE OHG, Hannover (DE)

(72) Inventors: Axel HAVERICH, Hannover (DE); Clemens MEYER-KOBBE, Sarstedt (DE); Michael HARDER, Hildesheim (DE)

(21) Appl. No.: 17/606,618

(22) PCT Filed: Apr. 27, 2020
(86) PCT No.: PCT/EP2020/061625

§ 371 Date: Oct. 26, 2021

(30) Foreign Application Priority Data
Apr. 29, 2019 (DE) 10 2019 110 982.5

(51) Int. Cl. A61M 60/88

(52) U.S. Cl. CPC A61M 60/88

(57) ABSTRACT

The invention relates to an intracorporeal guide component (1) for guiding lines belonging to medical devices inside a living being. The guide component (1) has a main body (2) comprising a line channel (3) for receiving and guiding the line. To secure the guide component (1) in the body of the living being more easily, with less damage to the tissue, according to the invention the main body (2) has, on its periphery, one or more contact surfaces (4, 4b) designed for interlocking and/or frictional contact with a bone structure of the living being. For example, the guide component (1) can be clamped between two costal arches of the living being, as an intercostal guide component (1), in order to securely hold and guide a line leading to the heart of the living being.

The invention relates to an intracorporeal guide component for guiding lines of medical devices within a living being. Here, the guide component has a base body with a line channel for receiving and guiding the line.

Guide components of this kind are used to guide, hold and fix cables, hoses, tubes or other lines of medical devices located in a body of a living being. For example, some implanted medical devices require a permanent power supply, which is generally made available outside the body and worn constantly on the body.

As a specific example, implanted artificial heart systems may be mentioned here. The connection, also called a driveline, between the artificial heart and an external controller/power supply is routed, starting from the controller, through the skin and into the body of the living being. The driveline generally contains a power supply line and a control or data line for the exchange, for example, of sensor and measurement data or control commands.

The guide component is provided in particular for transcutaneous lines. Such lines are routed through the skin of the living being. In such a use, provision is made that the exit point of the line is enveloped by skin, such that the latter can adhere to the line, preferably also to the guide component, and forms an infection barrier.

Manufacturers generally select line surfaces which promote adherence to the skin and which thus form a tight seal. For this purpose, however, there must be no tensile stress on the line, since movements of the line prevent it from adhering to the skin. The line can be fixed on the skin, for example using plastic wings that are anchored on a plaster. It should be noted in principle that the transmuscular and/or subcutaneous passage of the line is particularly susceptible to infection.

U.S. Pat. No. 10,105,537 B2 discloses a cable holder for pacemaker cables. The holder is fixed in an intercostal region, i.e. between two ribs of a living being, in or on muscle tissue, for example by being screwed on or sewn on. The purpose of the holder is to prevent an undesired change of position of the pacemaker cable and to isolate it electrically from the muscle through which the cable is guided.

US 2006/0025826 A1 discloses a subcutaneously implantable cardioverter-defibrillator with a telescopic line. The device has a housing with a guide channel in which the line is displaceably arranged in order to permit length compensation and freer positioning of the housing. To increase wearing comfort, the housing can be arranged intercostally parallel to and between the ribs of a patient. For this purpose, the housing can have an elongate and curved design.

US 2018/0272122 A1 discloses an implantable medical device that can be fastened to intercostal muscle tissue. For this purpose, the device has one or more elongate anchor structures which are inserted into the muscle tissue and thereby anchor the device at its predetermined intercostal position. The device can have a convex shape, so as to be able to be arranged flush on the muscle tissue.

US 2004/215303 A1 describes contacts for an electrical connection of biomedical implantable lines. These are biocompatible and electrically conductive contact connections that surround the conductor to be connected, for example in a ring shape.

US 2011/009933 A1 relates to an implantable electrical stimulation system with an implantable device that has a guide channel for one or more electrode lines.

US 2011/004286 A1 proposes an implantable cardio device with a line, in which the cardio device, for example designed as a pulse generator or defibrillator, is arranged in the thoracic cage or the abdominal wall of a patient and the line is routed to the heart. To stiffen and relieve the tensile stress on the line, a stiff line channel, for example with four openings for the conductor wires, is provided in some sections.

Against this background, the problem addressed by the invention is that of making available an improved guide component which can be fixed in the body of a living being more easily and in a manner that is gentle on the tissue. The guide component should in particular also be suitable for guiding transcutaneous lines.

The problem is solved by a guide component of the type in question in which the base body of the guide component has, on its circumference, one or more contact faces which are designed for form-fit and/or force-fit engagement on a bone structure of the living being.

Form-fit engagement is understood to mean a connection involving an interlocking of guide component and bone structure, in which the guide component and the bone structure, because of their shape, do not detach from each other. Here, the guide component and the bone structure are, as it were, “in the way” of each other, such that they support each other.

In the case of force-fit engagement, mutual displacement between guide component and bone structure is prevented on account of the static friction acting between the two components. As long as the static friction is not overcome by an external force, the component and the bone structure adhere to each other with a friction fit.

The base body can thus be held on the bone structure on the one hand by virtue of its shape, which for example in some sections exhibits a negative contour or an undercut of a bone structure, and on the other hand by virtue of its surface, which for example has a high coefficient of static friction. Of course, a combination of form-fit engagement and force-fit engagement is also conceivable.

The form fit can be made available, for example, by an at least partially curved shape of the guide component in cooperation with a rib curvature. Corresponding notches in the cranial and caudal faces of the guide component can improve the form fit.

According to the invention, a contact face, which takes up at least a portion of the surface of the base body, is designed for form-fit and/or force-fit engagement on the bone structure. The contact face can also be provided peripherally on a circumference of the base body or can be divided into a plurality of individual contact faces spaced apart from one another. It is therefore not necessary for the entire base body to be designed to bear on the bone structure.

A bone structure is understood to mean pressure-resistant and tension-resistant organs of the living being that are part of the skeleton of the latter. This can be a single bone or multiple bones or bone connections such as joints. The bone structure is preferably a rib structure, for example of a thoracic cage of the living being, wherein the rib structure can be formed by a single rib or a plurality of ribs, in particular two adjacent ribs.

The lines in question are in principle also rigid, but in particular flexible or at least partially flexible lines, i.e. pliable and/or elastic connecting channels that are suitable for carrying a medium or that are designed as power supply and/or data lines. For the power supply or data line, these can for example contain electrical conductors such as metallic wires, but also optical conductors such as optical fibers. The media-carrying lines are, for example, hoses, tubes or catheters which, for example, carry gases and gas mixtures such as air or liquids such as body fluids or medical solutions. A plurality of conductors can also be provided in a line encompassing them, for example a cable or hose with a plurality of electrical lines routed therein, such as a power supply line and a data line.

The line is generally connected to a medical device which can be designed, for example, as a control unit of an artificial heart system, as a dialysis machine or as a ventilator. In principle, a wide variety of medical devices are conceivable that can be connected to internal organs of a living being via lines. These do not have to be devices that are permanently worn on the body of the living being, but ones that are only regularly or irregularly attached to the line. However, the line itself is preferably routed permanently and not just temporarily in the living being concerned, such that the guide component is also intended for long-term use.

In this application, a living being is primarily understood as meaning human or animal living beings for which a medical treatment with a medical device of the type in question is possible.

The guide component according to the invention has a base body, which defines the essential three-dimensional extent and shape of the guide component. The base body can in principle have a regular or symmetrical basic shape such as that of a sphere, cuboid, cylinder, cube or truncated pyramid. However, irregular, polygonal and asymmetrical basic shapes are also conceivable. In one embodiment of the invention, the guide component has a trapezoidal base area, the narrower side of which can preferably be oriented dorsally when the guide component is in an installed state. Last but not least, the shape and the extent of the base body are based on the geometric properties of the intracorporeal destination site, in particular as regards the contact of the base body with a bone structure of the living being, as provided according to the invention.

Irrespective of the basic shape of the base body, the latter has a line channel for receiving and guiding the line. In the simplest case, such a line channel can be designed as a through-hole through the base body, such that a line channel with a circular guide cross section is formed. Of course, line channels with other guide cross sections, for example with rectangular or semicircular guide cross sections, are also conceivable. The line channel preferably leads through the interior of the base body in order to ensure a high degree of guidance stability. In other embodiments, however, an arrangement of the line channel on the circumference of the base body is also conceivable, for example in the form of a suitable groove or guide eyelets, which prevent the line from coming loose from the base body.

It should be noted that the word “a” or “an” in this application is not to be construed as meaning just one. Thus, in principle, a plurality of line channels can also be provided in a base body or a guide component in order to be able to hold and fix a plurality of different lines. In particular, a plurality of differently designed line channels can be provided which, for example, have different diameters.

A large number of primary shaping and reshaping manufacturing methods can be used to manufacture the base body. For example, the base body can be manufactured particularly simply by a casting or pressing method or with a high degree of individuality by an additive manufacturing method (for example a 3D printing method) or a modeling method. Structures that supplement the basic shape, for example the line channel, can already be taken into account in the manufacturing method, in order to manufacture a guide component with the desired geometric requirements in the smallest possible number of manufacturing steps.

With the guide component according to the invention, it is possible for the first time to be able to fix it, for example to clamp it, quickly and easily at the intended site in the body of the living being by engaging it on one or more bone structures. Thus, no additional connecting means, in particular screws or surgical threads, are required to attach the guide component. In addition, injuries to tissue, skin and bone are avoided or at least reduced, since the guide component does not necessarily have to be screwed or sewn onto them. It is therefore a guide component that is in principle implantable in a minimally invasive manner. In addition, the guide component engaging on the bone structure is arranged in a more stable manner, in particular in a more stable position, since it is supported on the comparatively solid or stationary bone structure.

By virtue of its being fixed with force-fit and/or form-fit engagement, the guide component can be of comparatively small size, such that, compared to solutions from the prior art, only a small tissue incision is required for the implantation of the guide component.

The guide component is in particular also suitable for the guiding of transcutaneous lines since, by bearing with form-fit and/or force-fit engagement on a bone structure, it can be positioned directly under the skin and held securely there.

In a particularly advantageous embodiment, the base body has at least two contact faces which are designed to bear with form-fit and/or force-fit engagement on bone structures of the living being and which lie opposite each other. The two contact faces are thus spaced apart from each other, specifically in such a way that the contact faces are formed in different planes or on different sides of the base body. For example, the contact faces can be formed on an upper and a lower side and/or on a left or right side of the base body. With regard to anatomical direction designations when the guide component is inserted into the body of the living being, the contact faces can be arranged, for example, on a cranial and a caudal plane and/or on a dorsal and a ventral plane of the base body. This embodiment is thus aimed in principle at ensuring that the guide component can be fixed between two mutually opposite bone structures of the living being. This for example promotes force-fit clamping between the bone structures. For this purpose, for example, the distance between the two contact faces can be dimensioned slightly greater than the distance between the bone structures provided for the engagement, such that an interference fit promotes the force-fit hold of the guide component between the bone structures. The oversize can also be generated by a curved design of a contact face, for example of the cranial or caudal contact face. Of course, the shape of the contact faces can also in principle be designed, for example as a negative contour or undercut of the bone structures provided for the engagement, in such a way that a form fit of the guide component on the bone structures is obtained. The embodiment having at least two mutually opposite contact faces improves the hold of the guide component at the intended location in the living being. In addition, positioning is made easier, since the plurality of contact faces with the respectively assigned bone structures define the positioning possibilities of the guide component more clearly.

In a particularly expedient embodiment, the guide component is designed as an intercostal guide component, of which the base body has a cranial contact face which is provided for form-fit and/or force-fit engagement on a cranial rib of the living being, and of which the base body has a caudal contact face which is provided for form-fit and/or force-fit engagement on a caudal rib of the living being. Provision is thus made that the guide component can be positioned between two adjacent or mutually opposite ribs, of which one rib forms the upper or cranial rib and the other rib forms the lower or caudal rib.

Of course, the guide component does not have to bear on the rib along the entire length thereof. In principle, provision is instead made that the guide component bears on a section of the respective rib. Advantageously, the cranial and caudal contact faces of the base body are adapted to the rib shape in the region of engagement of the guide component on the rib and for this purpose, for example, are of a concave design in order to be able to bear on the convex shape of the costal arch and thus generate a form fit. The height of the guide component corresponds to the distance separating the rib sections that are provided for the engagement. Alternatively or in addition, a slight interference fit of the guide component is expedient in order to be able to exert a sufficient clamping force on the adjoining rib sections. However, the chosen oversize should not be too high, in order not to generate a strong sensation of pressure or of a foreign body in the living being or even damage tissue, bone structure or guide component, and in order not to require too much effort when inserting the guide component. The advantage of the described embodiment as an intercostal guide component lies in the particularly high suitability of the rib structure as a contact partner for the contact faces of the guide component. The person implanting the guide component can decide relatively freely and individually between which costal arches and at which position the guide component is to be placed, since the rib structure is relatively regular and several pairs of ribs are possible as contact partners for the guide component. This embodiment is also advantageous for medical applications relating to the heart, since the guide component can be used to hold the line close to the heart in the thoracic cage of the living being. As a result, compressive and tensile forces acting on the line are taken up by the guide component close to the heart, and the parts of the medical device that are located on the heart are relieved of mechanical stress.

This embodiment of the guide component, adapted to the rib structure of the living being, is also particularly suitable for transcutaneous lines, since the guide component can be placed on the ribs close behind the passage of the line through the skin, thereby promoting the adherence of the line and/or of the guide component.

According to an advantageous embodiment, the guide component is formed in several parts from a plurality of guide component elements. Particularly preferably, the guide component is designed here in two parts, i.e. from two guide component elements. In particular, the guide component can here be composed of two halves. The dividing plane of the guide component lies, for example, in the plane of the line channel. Thus, a part of the line channel is integrally formed on each of the guide component elements, such that the line channel is closed only when the guide component elements are put together. This makes it possible to arrange the guide component particularly gently around a line that has already been routed in the body of the living being, without having to thread the line through the closed guide component from the end of the line. This also affords the advantage that it can be retrofitted, since the guide component can still be implanted even with lines that have already adhered. In practice, it is sometimes not even possible to thread the line through the guide component, since the line may have a plug with a considerably wider cross section than the line. The provision of a larger line channel through which the line and the plug could be passed would entail disadvantages as regards securely holding and fixing the line in the line channel. By contrast, since the guide component is made up of several parts, in particular with a dividing plane running through the line channel, the latter can be optimally adapted to the cross section or diameter of the line.

It is advantageous if the guide component elements of the multi-part guide component have connecting means for producing a connection, in particular a releasable connection, to one another. These are in particular connecting means which are preferably formed directly onto the guide component elements, in particular formed in one piece with them, such that no additional, separate connecting means such as screws have to be kept ready. In particular, they are connecting means for producing a plug-in, latching or clip connection. For example, one guide component element can have a latching lug, and another guide component element can have a latching opening in which the latching lug can be latched. By means of the connecting means described above, the guide component elements can be put together, and also detached from one another, particularly quickly and easily and in particular without tools, for example in order to replace the guide component. The guide component elements can be positioned around the line that is to be held, and they can then be connected to one another simply by closing the latching connection, without the line having to be passed over its entire length through the line channel of the already assembled guide component elements. The at least one connecting means is preferably arranged on a ventral plane or surface of the guide component. In this way, better accessibility and maneuverability of the connecting means can be ensured in the case of an implantation that is carried out from the ventral aspect of the living being.

The base body of the guide component advantageously has a basic shape curved around a cranial-caudal central axis of the base body. In the inserted state of the guide component, the central axis thus runs in the cranial to caudal direction, or vice versa. The curve follows in particular the curve of a bone structure on which a contact face of the guide component bears. In the case of a rib for example, the latter, viewed horizontally, runs in an arc, which the guide component can follow in terms of its shape. Thus, by virtue of the curved basic shape, the guide component is better adapted to the profile of the rib or of a comparable bone structure, such that the hold of the guide component is further improved. It should be noted here that the curved profile of the rib, viewed in a horizontal perspective, is not to be equated with the curved cross section of the rib, which is taken into account in the guide component, for example by a concave contour of the contact faces.

According to an expedient embodiment, the line channel runs between a ventral surface of the base body and a dorsal surface of the base body. The line is thus routed on the shortest path from an internal organ toward the human skin on the ventral or dorsal aspect. Moreover, this feature supports embodiments in which the guide component is positioned between a cranial and a caudal bone structure, such that the inlet or outlet of the guide channel is not blocked or impaired by the bone structure.

It is advantageous if the line channel for receiving and guiding the line is designed as an inclined bore through the base body. In this way, when the guide component is inserted into the body, the line channel runs neither strictly vertically nor horizontally. In such an embodiment, there are particularly few or no observable line diversions, such that the intended tensile stress relief is maximized. In addition, there is no catching and in particular no kinking of the line at the inlet or outlet of the line channel, such that damage to the line while it is being guided through the guide component is prevented. In this way, a cable break or cable kinking is reliably avoided. In the case of a transcutaneous line, this also allows the extracorporeal section of the line to be routed close to the skin. The extracorporeal section and also the intracorporeal section of the line are not curved or are only slightly curved, such that no or only slight forces are exerted on the exit site. This promotes rapid healing and adherence of the tissue to the line and/or the guide component.

The line channel advantageously has a cross-sectional constriction in parts. Alternatively or in addition, a fixing structure of the base body protrudes into the line channel. A fixing structure of this kind can be formed, for example, by knobs, material tips, transverse webs or comparable material projections. In this way, the line is fixed, in a targeted manner or as and when necessary, and relieved of tensile stress within the line channel. This reduces or prevents undesired displacements of the line and increases the tensile stability of the line.

The base body preferably has at least one fastening structure formed in one piece with the base body. The fastening structure can be provided in particular on the lateral surfaces, that is to say in particular on the ventral or dorsal surface of the guide component, in order to facilitate the fixing of the guide component. The fastening structure can, for example, be arranged around the cable channel, on a peripheral face of the cable channel. Such a fastening structure can, for example, be in the form of one or more eyelets so that, if necessary, the guide component can additionally be sewn and thus fixed to surrounding tissue, for example muscles, fasciae, skin or fatty tissue, or also to surrounding or adjacent bone structures. Other fastening structures are also conceivable, such as clip or spring elements. Overall, the hold of the guide component at the intended position in the body of the living being is improved by the fastening structure, and the fact that the fastening structure is integral with the base body simplifies the production of the guide component.

In an expedient embodiment, the base body has a ventral fastening rim in the region of the line channel. In the case of a line that is guided transcutaneously, the fastening rim serves for fixing the skin to the guide component such that a tight seal is permitted.

In an advantageous embodiment, the base body has at least one fastening opening. Fastening means such as threads or screws can if necessary be passed through such fastening openings, which are preferably through-bores, in order to additionally connect the base body of the guide component to a tissue or bone structure, for example in order to screw the guide component onto a bone structure. Such an additional connection is useful if, for example, the expected mechanical loads on the line or on the guide component may briefly exceed the limit force of the force-fit and/or form-fit engagement of the guide component on the bone structure and if the hold of the guide component should therefore be additionally supported.

In the present case, a fastening structure, holding structure or fixing structure is understood to mean a material addition, while a fastening opening entails a material recess.

It is expedient if the one or more contact faces are designed as a groove or grooves running at least in sections on the circumference of the base body. This in principle corresponds substantially to a concave embodiment of the contact faces, wherein the shape of the contact face in question defines a groove base with corresponding side walls. Such a groove can be provided on the entire circumference of the base body, such that a freer orientation of the guide component is permitted during its implantation. However, two mutually opposite contact faces, for example, can also be designed as a groove. The design of the contact faces as a groove or grooves simplifies the production of the guide component, improves the fit thereof, facilitates the pressing or clamping of the guide component on bone structures, and increases the individual freedom in positioning the guide component. A particular advantage of the groove or grooves is that vessels located at the bone structure, such as blood vessels, for example the intercostal artery, are accommodated in the groove without being pinched, such that the blood circulation at the bone structure is not impaired by the guide component.

At least one holding structure is preferably arranged on the groove base of the groove or grooves, in particular being formed in one piece with the base body. This holding structure can, for example, be in the form of molded knobs or crosspieces. The knobs can be designed, for example, as pointed knobs with a pyramidal or conical basic shape. This holding structure additionally secures the guide component against displacement relative to the bone structure, for example by virtue of the holding structure increasing the surface interacting between the two contact partners and thus increasing the existing frictional forces.

The surface of the guide component should have the greatest possible roughness, at least on the contact faces, but preferably over the entire guide component, for example by provision of a mechanically or chemically roughened surface. This permits better incorporation of the guide component and promotes the positional stability on the ribs. The roughness on individual surfaces, for example on the caudal contact face, can also be increased by means of points, for example conical or pyramidal material projections. The roughness increases the coefficient of static friction and thus the achievable frictional connection of the guide component with adjacent structures.

According to an advantageous embodiment, the diameter of the line channel corresponds to the diameter of the line that is to be received by the line channel. The associated flush guidance of the line in the line channel prevents, among other things, the admission of liquids or tissue into the line channel, such that tissue cannot become pinched and liquid cannot accumulate. This therefore also reduces the risk of injury and inflammation on the guide component. In addition, the frictional reception of the line in the line channel already ensures that the position of the line is fixed against axial displacement.

In an expedient embodiment, the guide component is made from a biocompatible material, in particular from a biocompatible metal or plastic. A high degree of material compatibility is achieved in this way, at the same time with a high degree of stability and long service life of the guide component.

The invention is explained in more detail below on the basis of exemplary embodiments and with reference to the accompanying schematic drawings, in which:

FIG. 1 shows a dorsal plan view of a two-part guide component in the assembled state;

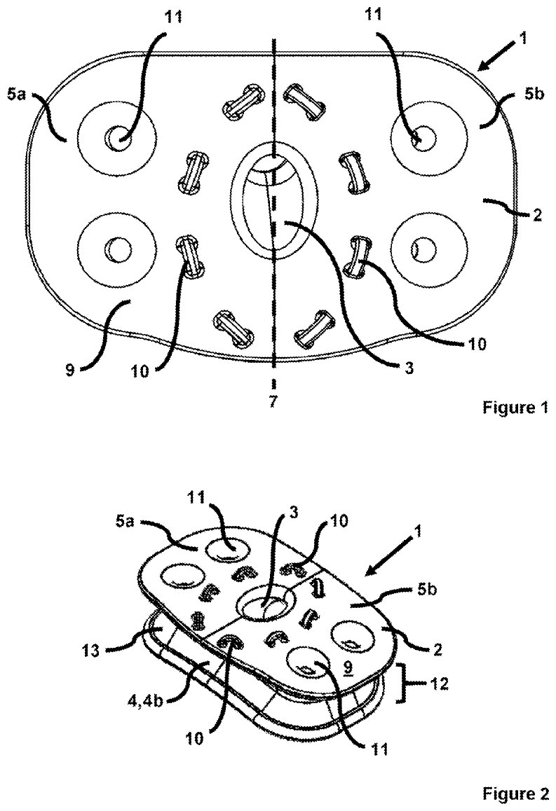

FIG. 2 shows a perspective dorsal view of the two-part guide component in the assembled state;

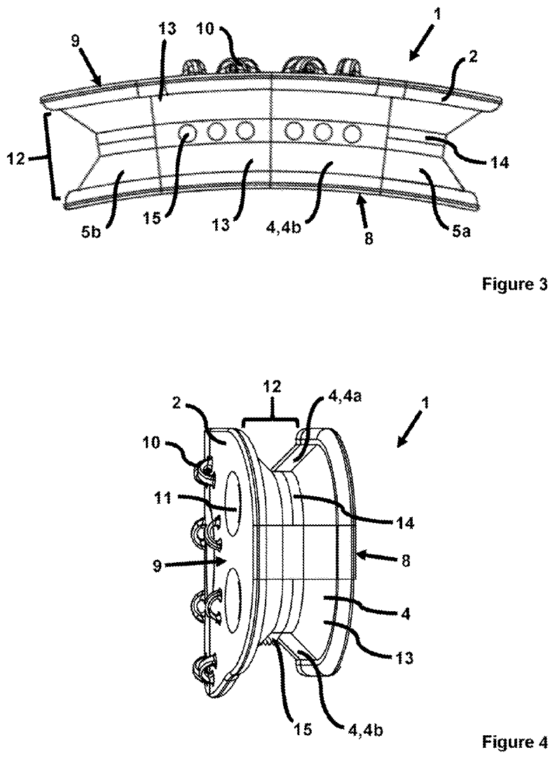

FIG. 3 shows a caudal view of the two-part guide component in the assembled state;

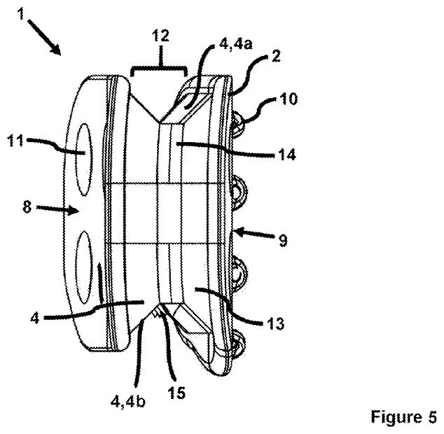

FIG. 4 shows a side view of the two-part guide component in the assembled state;

FIG. 5 shows a further side view of the two-part guide component in the assembled state;

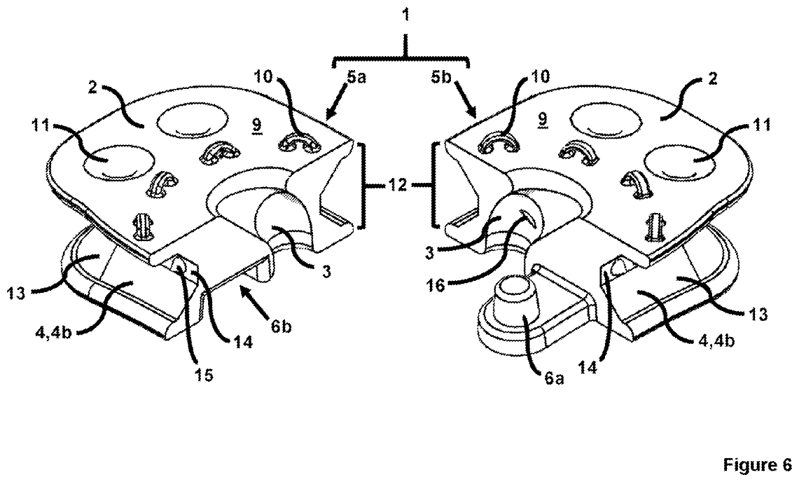

FIG. 6 shows a perspective dorsal view of the two-part guide component, with guide component elements separated from each other;

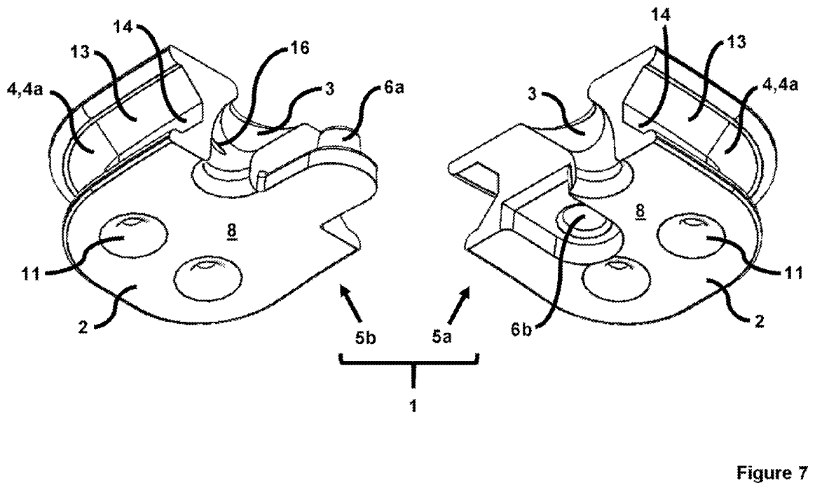

FIG. 7 shows a perspective ventral view of the two-part guide component, with guide component elements separated from each other.

FIG. 1 shows a multi-part embodiment, specifically a two-part embodiment, of the guide component 1 according to the invention, which is composed of two guide component elements 5a, 5b releasably connected to each other. The guide component 1 is intended to guide, hold and fix one or more lines (not shown) of medical devices in a body of a living being. For this purpose, the guide component 1 has contact faces 4 (shown in the other figures) which are designed for form-fit and/or force-fit engagement on a bone structure of the living being. This makes it possible for the guide component 1 to be fixed quickly and easily at the intended site in the body of the living being by engaging it on one or more bone structures, for example by being able to clamp it. The guide component 1 shown is particularly intended to be arranged as an intercostal guide component 1 between two adjacent or mutually opposite ribs of a living being. With respect to an implantation state of the guide component 1, the view in FIG. 1 is a dorsal view of the guide component, i.e. facing the back of the living being 1. The figure shows the base body 2, of approximately rectangular cross section, and a line channel 3 which is designed as an inclined through-bore and extends substantially centrally through the base body 2. On account of the inclined bore, there are very few or no observable line deflections, and therefore the intended tensile stress relief is maximized. In addition, there is no catching and in particular no kinking of the line at the inlet or outlet of the line channel 3, such that damage to the line during the guidance through the guide component 1 is prevented. In the embodiment shown, the line channel 3 runs from a ventral surface 8 of the base body 2 to a dorsal surface 9 of the base body 2. A cranial-caudal central axis 7 of the base body 2 runs through the base body 2, around which central axis 7 the base body 2 is slightly curved, as can be seen in particular in FIG. 3. FIG. 1 also shows fastening structures 10 which are integral with the base body 2 and are in the form of a total of eight eyelets which are arranged on the dorsal surface 9 of the base body 2 and, for example, allow the guide component 1 to be sewn onto a bone, skin or tissue structure. It is thereby possible, if necessary, to achieve an additional hold of the guide component 1 at the intended position in the body of the living being. Furthermore, FIG. 1 shows a total of four fastening openings 11 in the base body 2, through which, for example, threads or screws can be guided in order to be able to fix the guide component 1 to a bone or tissue structure of the living being by means of a screw connection. It is thereby possible, if necessary, to achieve an additional hold of the guide component 1 at the intended position in the body of the living being. The guide component 1 has on its circumference, in the region of the caudal contact face 4b, a slight curvature that leads to a slight interference fit of the guide component 1 and, in the inserted state of the guide component 1, increases the contact pressure acting on the adjacent bone structure.

FIG. 2 shows a perspective dorsal view of the guide component 1. The constituent parts of the guide component 1 that are located or visible on the dorsal surface 9 of the base body 2 have already been explained with reference to FIG. 1. Also shown in FIG. 2 is the contact face 4 with which the base body 2, in the implanted state of the guide component 1, bears with form-fit and/or force-fit engagement on a bone structure of the living being. It can be seen that the contact face 4 is arranged as a concave contact face 4 on the circumference of the guide component 1 between the dorsal surface 9 and the ventral surface 8. By virtue of the concave shape, the guide component 1 is particularly suitable for convex bone structures or bone sections such as the ribs. In the view in FIG. 2, the contact face 4 is a caudal contact face 4b of the guide component 1. If the guide component 1 is inserted, for example, between two costal arches, the caudal contact face 4b lies on a cranial section of the upper costal arch facing toward the head. In the embodiment shown, the contact face 4 is designed as a peripheral groove 12 on the circumference of the guide component, having groove side walls 13 and a groove base 14 shown in the further figures. By means of the contact face 4 designed as groove 12, any vessels such as blood vessels on the bone structure can be received without being pinched, such that the blood circulation at the bone structure is not impaired by the guide component 1. By virtue of the fact that the groove 12 is formed circumferentially, i.e. without interruption continuously defines the circumferential shape of the guide component 1, the latter can be positioned relatively freely on a bone structure, for example in a slightly inclined position or also at a place where it is surrounded or enclosed by several bones. In principle, however, it is just as conceivable to provide the groove 12 only in sections or to provide several grooves 12.

FIG. 3 shows a caudal view of the two-part guide component 1 in the assembled state of the two guide component elements 5a, 5b. This view shows the contact face 4 with which the base body 2, in the implanted state of the guide component 1, rests with form-fit and/or force-fit engagement on a bone structure of the living being. It can be seen that the contact face 4 is arranged as a concave contact face 4 on the circumference of the guide component 1 between the dorsal surface 9 and the ventral surface 8. By virtue of the concave shape, the guide component 1 is particularly suitable for convex bone structures or bone sections such as the ribs. In the view in FIG. 3, the contact face 4 is a caudal contact face 4b of the guide component 1. If the guide component 1 is for example inserted between two costal arches, this caudal contact face 4a lies on a cranial section of the upper costal arch facing toward the head. In the embodiment shown, the contact face 4 is designed as a peripheral groove 12 on the circumference of the guide component 1, having groove side walls 13 and a groove base 14. By means of the contact face 4 designed as groove 12, any vessels such as blood vessels on the bone structure can be received without being pinched, such that the blood circulation at the bone structure is not impaired by the guide component 1. By virtue of the fact that the groove 12 is formed circumferentially, i.e. without interruption continuously defines the circumferential shape of the guide component 1, the latter can be positioned relatively freely on a bone structure, for example in a slightly inclined position or also at a place where it is surrounded or enclosed by several bones. In principle, however, it is just as conceivable to provide the groove 12 only in sections or to provide several grooves 12. On the caudal contact face 4b, holding structures 15 are formed in the groove bottom 14, which holding structures 15 are in the form of knobs which protrude from the groove base 14 and which are integral with the base body 2. These secure the guide component 1 additionally against displacement in relation to the bone structure, by means of the fact that the holding structure 15 increases the surface interacting between the contact face and the bone structure and thus increases the existing frictional forces.

FIGS. 4 and 5 each show a side view of the two-part guide component 1, in which once again the design of the contact face 4 as a circumferential groove 12 with groove side walls 13 and a groove base 14 is apparent. The contact face 4 shown transitions into the caudal contact face 4b on the lower side in the picture and transitions into the cranial contact face 4a on the upper side in the picture.

FIGS. 6 and 7 show in succession a dorsal view and a ventral view of the two-part guide component 1 with guide component elements 5a, 5b separated from each other. Both views show a connecting means for connecting the two guide component elements 5a, 5b, namely in the form of a latching lug 6a of the guide component element 5a and a corresponding latching opening 6b of the guide component element 5b. In an assembled state of the guide component 1, the latching lug 6a engages in the latching opening 6b of the guide component element 5b. By plugging them together, i.e. inserting and locking the latching lug 6a in the latching opening 6b, the two guide component elements 5a, 5b can be connected to each other in a reliable but releasable manner. It can also be seen that the dividing plane of the guide component 1 for the separation into the guide component elements 5a, 5b runs through the line channel 3. Thus, one part of the line channel 3 is integrally formed on the one guide component element 5a as a channel with a semicircular cross section, and the other part of the line channel 3 is integrally formed on the other guide component element 5b as a channel with a semicircular cross section. The line channel 3 is closed by joining the two guide component elements 5a, 5b together. In this way, a line already routed in the body of the living being can be enclosed by the line channel 3 of the guide component 1.

In the wall of the line channel 3, there is also a fixing structure 16, which protrudes into the line channel 3. The fixing structure 16 is formed, for example, by an elongate material projection. In this way, the line is fixed, in a targeted manner or as and when necessary, and relieved of tensile stress within the line channel 3. This reduces or prevents undesired displacements of the line and increases the tensile stability of the line.

The guide component 1 is preferably made from a metal or plastic that is biocompatible and thus eminently compatible with the surrounding tissue and bone structures of the living being. The guide component 1 can have a certain basic elasticity, in order to be able to be fastened to a bone structure, for example by being clamped thereon, without too much effort. However, the guide component 1 should also have sufficient basic stability in order to ensure a reliable hold of the line.

The anatomical directional designations used in this application, such as cranial, caudal, ventral and dorsal, are merely illustrative and are provided in order to promote the spatial concept of the subject matter of the invention. The directional designations should not be understood as restrictive in the sense that the guide component according to the invention can be inserted into the body of a living being only in one orientation and position. Thus, it is in principle conceivable that, for example, the ventral and dorsal sides of the guide component are interchangeable, likewise the cranial and caudal sides. Similarly, by pivoting the guide component 1 through 90° for example, what was previously a lateral contact face of the guide component could become a cranial or caudal contact face.

1. An intracorporeal guide component, comprising:
a base body with a line channel for receiving and guiding one or more lines; and
one or more contact faces positioned on a circumference of the base body which are designed for form-fit and/or force-fit engagement on a bone structure of a living being.

2. The intracorporeal guide component as claimed in claim 1, wherein the one or more contact faces comprises at least two mutually opposite contact faces designed for form-fit and/or force-fit engagement on bone structures of the living being.

3. The intracorporeal guide component as claimed in claim 2, wherein the guide component is designed as an intercostal guide component of which the base body comprises a cranial contact face which is configured for form-fit and/or force-fit engagement on a cranial rib of the living being, and wherein the base body comprises a caudal contact face which is configured for form-fit and/or force-fit engagement on a caudal rib of the living being.

4. The intracorporeal guide component as claimed in claim 1 wherein the intracorporeal guide component is formed from several parts from a plurality of guide component elements.

5. The intracorporeal guide component as claimed in claim 4, wherein the guide component elements comprise one or more connectors for connecting to each other.

6. The intracorporeal guide component as claimed in claim 1 wherein the base body has a basic shape curved around a cranial-caudal central axis of the base body (2).

7. The intracorporeal guide component as claimed in claim 1 wherein the one or more line channels runs between a ventral surface of the base body and a dorsal surface of the base body.

8. The intracorporeal guide component as claimed in claim 1 wherein the one or more line channels are designed as an inclined bore through the base body.

9. The intracorporeal guide component as claimed in claim 1 wherein the one or more line channels has a cross-sectional constriction in parts, and/or wherein a fixing structure of the base body (2) protrudes into one of the one or more line channels.

10. The intracorporeal guide component as claimed in claim 1 wherein the base body has at least one fastening structure formed in one piece with the base body.

11. The intracorporeal guide component as claimed in claim 1 wherein the base body has at least one fastening opening.

12. The intracorporeal guide component as claimed in claim 1 wherein the one or more contact faces are designed as a groove or grooves running at least in parts on the circumference of the base body.

13. The intracorporeal guide component as claimed in claim 12, further comprising at least one holding structure arranged on a groove base of the groove or grooves.

14. The intracorporeal guide component as claimed in claim 1 wherein at least one of the one or more line channels has a diameter which corresponds to a diameter of a line that is to be received by the at least one line channel.

15. The intracorporeal guide component as claimed in claim 1 wherein the intracorporeal guide component is made of a biocompatible metal or biocompatible plastic.

16. The intracorporeal guide component of claim 5 wherein the one or more connectors form a releasable connection.

17. The intracorporeal guide component of claim 13 wherein the at least one holding structure is formed in one piece with the base body.
